(19) United States
(12) Patent Application Publication
(10) Pub. No.: US 2006/0073068 A1
(43) Pub. Date: Apr. 6, 2006

(54) Composition for disinfecting surfaces

(75) Inventor: Bruce Kelly Krimpton Crosby, North Vancouver (CA)

Correspondence Address: Clifford W. Vermette, Vermette & Co.;Suite 230-200, Granville Street, Box 40 Granville Square, Vancouver, BC, V6C 1S4, CA

(73) Assignee: Ocean Air Systems Ltd.

(21) Appl. No.: 10/954,221

(22) Filed: Oct. 1, 2004

(51) Int. Cl. A61L 2/22

(52) U.S. Cl. 422/28; 424/405; 424/616; 424/770

(57) ABSTRACT

A disinfectant composition, which has an aqueous solution of hydrogen peroxide, an emulsifier and cedar leaf oil. The cedar leaf oil provides a residual long term disinfecting effect following the initial disinfecting of the hydrogen peroxide and then its decomposition. Application of the disinfectant is accomplished by first nebulizing the disinfectant solution. The nebulizing produces selected particle sizes to optimize the contact time with a surface to be treated.

### FIELD

The present invention relates to a composition for disinfecting surfaces and a method of application for same.

### BACKGROUND

Disinfectants are chemical substances used to kill viruses and microbes (germs), such as bacteria and fungi. Some disinfectants are able to only partially sterilize. The most resistant pathogens are bacterial spores but some viruses and bacteria are also highly resistant to many disinfectants. Disinfectants are potentially harmful and even possibly toxic to humans or animals. They are used in hospitals, dental surgeries, kitchens and bathrooms to kill infectious organisms. The choice of disinfectant depends on the particular situation. Some disinfectants have a wide spectrum in that they kill nearly all microorganisms.

Examples of the types of disinfectants are peroxygen compounds, chlorine compounds, phenolics, and quaternary ammonium compounds. Peroxygen compounds include hydrogen peroxide, peroxymonosulphuric acid, and persulphates, all of which are used destroy free and complexed cyanides. Chlorine is used to disinfect water of water borne diseases. Phenol and phenolics form the active ingredient in most mouthwashes and in disinfectant soap and handwashes. It was previously referred to as carbolic acid. Quaternary ammonium compounds are a large group of related compounds that have been used as a low level disinfectant effective against bacteria but not against spores or viruses. The rate at which these compounds operate is often very slow and some leave a residue or emit volatile organic compounds.

Hydrogen peroxide is a strong oxidizing agent and a weak acid in water solution. It decomposes in the presence of light, metal ions and oxidizable organic materials into water and oxygen. A small amount of stabilizer such as acetanilide is normally added to the solution to retard the decomposition. Its oxidizing properties are used in bleaching and deodorizing for textiles and in the manufacture of paper. It is also used medicinally as an antiseptic, in water sewage treatment, mining, electronics, food and the cosmetic industry. Aqueous solutions of hydrogen peroxide have been used as a disinfectant; however, their use has been limited due to their instability. Moreover, hydrogen peroxide solution is difficult to apply. Given the wide spectrum of antimicrobial activity, the fact that hydrogen peroxide destroys microorganisms not by intoxication but by oxidation and that after use it remains in the form of water, it seems like a candidate as an ideal disinfectant but for these problems.

U.S. Pat. No. 3,954,974 issued to Herzog, et al. discloses a disinfectant which is an oil in water emulsion with hydrogen peroxide. Herzog et al. found that in such a solution the hydrogen can be stored without decomposition over a reasonable time and still remain effective in disinfecting the surface of a human body part. The effectiveness of this solution when applied to hard surfaces is not discussed. Furthermore, the disinfectant of Herzog has a relatively short period of efficacy (i.e. it does not have a long term anti-microbial effect) and, therefore, is really only useful for a limited number of applications (e.g. disinfecting skin or body parts prior to treatment or surgery).

U.S. Pat. No. 6,114,298 issued to Petri et al. discloses a microemulsion suitable for disinfecting a surface which has a surfactant, a bleach, a radical scavenger, solvents and droplets of an essential oil in an aqueous solution having a particle size of less than 0.1 microns.

In applying disinfectants to hard surfaces such as furniture, floor and walls of a hospital room, wiping on the solution is not effective in reaching the ceiling, around shielded and concealed areas, on fabric covered furniture and on blankets, curtains, mattresses, insulation and various other textiles. Petri discloses utilizing spray-type dispensers to apply the disinfectant. However, no details are given as to particle size which is an important determinant of disinfecting effectiveness.

Accordingly, it is an object to provide a particularly effective oil-in-water emulsion for use in disinfecting hard surfaces that has a long residual disinfecting effect.

It is a further object of the invention to provide a method of application to hard surfaces that reaches all shielded areas and covers the surface of soft fabrics in a room.

### SUMMARY OF THE INVENTION

According to the invention there is provided a disinfectant composition which includes an aqueous solution of hydrogen peroxide, an emulsifier and cedar leaf oil.

Preferably, the concentration of hydrogen peroxide is in the range of 0.5% to 12% by weight.

The pH of the disinfectant composition may be in the range of 2.6 to 3.4.

Advantageously, the emulsifier is a denatured alcohol. It may also be soap or a surfactant and spreading agent.

In another aspect of the invention there is provided a A method of coating surfaces of an object with a disinfectant composition which comprises nebulizing the composition into aerosolized particles.

The size of the particles nebulized may be varied to optimize contact time and dispersion.

### DETAILED DESCRIPTION

The following are examples of disinfectant compositions according to the present invention:

### EXAMPLE 1

90 g of water, 10 g. of 25% hydrogen peroxide, 5 g. of denatured alcohol, and 2.5 g. of cedar leaf oil mixed together. Cedar leaf oil is a volatile oil from the branches and leaves of Thuja occidentalis L. Pinaceae.

One litre of distilled water with 1 litre of an aqueous solution containing 175 g. of 30% hydrogen peroxide are mixed with the water and denatured alcohol and then heated to 40 degree C. The cedar leaf oil is then added slowly while stirring the mixture. The resultant emulsion is cooled to room temperature.

### EXAMPLE 2

85 g. of water, 10 g. of 25% hydrogen peroxide, 14 g. of a surfactant and spreading agent and 3.0 g. of cedar leaf oil. A suitable surfactant and spreading agent is Tween 20 (trademark).

### EXAMPLE 3

80 g. of water, 10 g. of 30% hydrogen peroxide, 7 g. of dishwater detergent, 2.6 g. of cedar leaf oil.

Examples 2 and 3 are made in a similar manner to Example 1. In this manner one obtains about 1 kg of a disinfectant having a pH of between 2.6 and 3.4 and a hydrogen peroxide concentration of 3.8%. This concentration can vary between about 0.5% to 12%.

The combined effect of the hydrogen peroxide, which loses its effectiveness as a disinfectant over time, and the long term disinfecting effect of the cedar leaf oil achieves a strong effect over a prolonged period of time. Cedar leaf oil is an effective antiviral agent and stimulant of the immune system. It may also be used to treat other infectious and/or immune diseases as well as neoplasic pathologies.

In applying the disinfecting composition to hard surfaces it is desirable to first nebulize the liquid into a desired particle size, which is determined by the required contact time. The range of particle sizes is 2 to 20 microns. The nebulized liquid is sprayed from a wand or vacuum outlet in order to fog a room or hallway. The nebulized particles float in the air and can reach shielded areas or can deposit on and disinfect insulation, textiles, furniture, walls and ceilings. Changing a dome plate in the nebulizing nozzle of commercially available nebulizers can vary the particle size. The particle size can in this way be selected in order to provide the desired contact time required to kill the pathogens and germs present and to maximize the dispersion of the disinfectant.

Accordingly, while this invention has been described with reference to illustrative embodiments, this description is not intended to be construed in a limiting sense. Various modifications of the illustrative embodiments, as well as other embodiments of the invention, will be apparent to persons skilled in the art upon reference to this description. It is therefore contemplated that the appended claims will cover any such modifications or embodiments as fall within the true scope of the invention.

I claim:

1. A disinfectant composition comprising:
(a) an aqueous solution of hydrogen peroxide;
(b) an emulsifier; and
(c) cedar leaf oil.

2. A composition according to claim 1, wherein the concentration of hydrogen peroxide is in the range of 0.5% to 12% by weight.

3. A composition according to claim 1, wherein the pH of the disinfectant composition is in the range of 2.6 to 3.4.

4. A composition according to claim 1, wherein the emulsifier is a denatured alcohol.

5. A composition according to claim 1, wherein the emulsifier is soap.

6. A composition according to claim 1, wherein the emulsifier is a surfactant and spreading agent.

7. A method of coating surfaces of an object with a disinfectant composition according to claim 1, comprising nebulizing the composition into aerosolized particles having a diameter in the range of 2 to 20 microns.

8. The method of claim 7, including varying the size of particles nebulized to optimize contact time and dispersion.

9. The method of claim 7, including entraining the nebulized particles in an air stream to deliver them to an area designated for disinfecting in the form of a fog or fine spray.
